(19) United States
(12) Patent Application Publication
(10) Pub. No.: US 2009/0203726 A1
(43) Pub. Date: Aug. 13, 2009

(54) SUBSTITUTED TETRAHYDROQUINOLINES AS ANTIBACTERIAL AGENTS

(75) Inventor: Roger FRECHETTE, Reading, MA (US)

Correspondence Address: MCCARTER & ENGLISH, LLP BOSTON, 265 Franklin Street, Boston, MA, 02110, US

(73) Assignee: MAXTHERA INC., Beverly, MA (US)

(21) Appl. No.: 12/324,496

(22) Filed: Nov. 26, 2008

Related U.S. Application Data
(60) Provisional application No. 60/991,535, filed on Nov. 30, 2007.

(51) Int. Cl. A61K 31/473; C07D 221/06; A61P 31/04

(52) U.S. Cl. 514/290; 546/79

(57) ABSTRACT

A method of treating a subject for a bacterial infection includes administering to a subject in need of treatment for a bacterial infection an effective amount of a compound represented by structural formula (I-a), or a pharmaceutically acceptable salt, solvate, or hydrate thereof.

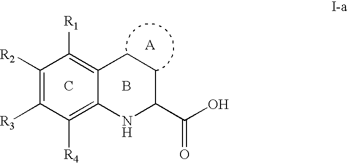

The variables in structural formula (I-a) are described herein.

### RELATED APPLICATION

This application claims priority to U.S. Provisional Application No. 60/991,535, Attorney Docket No. NPZ-008-1, filed Nov. 30, 2007, entitled “SUBSTITUTED TETRAHYDROQUINOLINES AS ANTIBACTERIAL AGENTS.” The contents of any patents, patent applications, and references cited throughout this specification are hereby incorporated by reference in their entireties.

### GOVERNMENT SUPPORT

This invention was made with government support from the National Institutes of Health. The government has certain rights in the invention.

### TECHNICAL FIELD

The present invention relates to compositions which target MurA and methods and uses thereof.

### BACKGROUND OF THE INVENTION

In the last century, antibiotics were developed that led to significant reductions in mortality. Unfortunately, widespread use has led to the rise of antibiotic resistant bacteria, e.g., methicillin resistant Staphyloccocus aureus (MRSA), vancomycin resistant enterococci (VRE), and penicillin-resistant Streptococcus pneumoniae (PRSP). Some bacteria are resistant to a range of antibiotics, e.g., strains of Mycobacterium tuberculosis resist isoniazid, rifampin, ethambutol, streptomycin, ethionamide, kanamycin, and rifabutin. In addition to resistance, global travel has spread relatively unknown bacteria from isolated areas to new populations. Furthermore, bacteria used in biological weapons can not be easily treated with existing antibiotics.

Infectious bacteria employ the peptidoglycan biosynthesis pathway, and in particular, depend on MurA (phosphoenolpyruvate:UDP-N-acetyl-D-glucosamine 1-carboxyvinyltransferase, EC 2.1.5.7) to catalyze the transformation of uridine diphosphate-N-acetyl-D-glucosamine and phosphoenolpyruvate into uridine diphosphate-N-acetyl-3-O-(1-carboxyvinyl)-D-glucosamine. MurA is conserved across both Gram positive and Gram negative bacteria, but is not present in mammalian systems, and is thus a desirable and selective target for new medications.

Therefore, there is a need for new medications that target MurA, whereby infections from bacteria dependent on MurA can be treated.

### SUMMARY OF THE INVENTION

The present invention relates to certain substituted tetrahydroquinoline MurA inhibitors as antibacterials. The disclosed compounds have antibiotic activity against bacteria, including drug-resistant bacteria. Based on this, compounds that inhibit MurA, methods of treatment with the disclosed MurA inhibitors, and pharmaceutical compositions comprising the disclosed MurA inhibitors, and methods for screening for MurA inhibitors are provided herein.

In one aspect, the invention provides a method of treating a subject for a bacterial infection includes administering to a subject in need of treatment for a bacterial infection an effective amount of a compound represented by structural formula I-a:

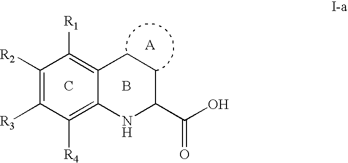

and pharmaceutically acceptable salts, solvates, hydrates, enantiomers, stereoisomers, rotamers, tautomers, diastereomers or racemates thereof.

The invention is useful for treating (therapeutically or prophylactically) bacterial infections, particularly infections caused by bacteria that depend on the peptidoglycan biosynthesis pathway, and more particularly, infections caused by bacteria that express the MurA enzyme. Furthermore, it can be useful against bacteria that have developed antibiotic resistance, especially multiple drug resistant strains, because it is believed to act through a different mechanism than existing, widely used antibiotics.

### DETAILED DESCRIPTION OF THE INVENTION

The invention is generally related to methods, compounds, and pharmaceutical compositions for treating and preventing bacterial infections. In particular, the invention relates to substituted tetrahydroquinoline derivatives that are MurA inhibitors.

In one embodiment, the MurA inhibitor of the method is represented by structural formula I-a:

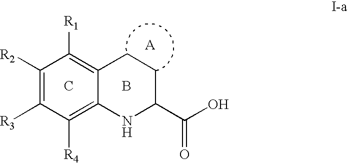

and pharmaceutically acceptable salts, solvates, hydrates, enantiomers, stereoisomers, rotamers, tautomers, diastereomers or racemates thereof; wherein

ring A is a 5 or 6 membered cycloalkyl or cycloalkenyl group, optionally substituted with halogen or optionally halogenated C₁-C₃ alkyl or alkoxy;

the variables R₁, R₂, R₃ and R₄ are each, independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C₃ to C₇ cycloalkyl, nonaromatic heterocycle, C₁ to C₄ alkyl, C₁ to C₄ alkoxy, C₁ to C₄ hydroxy alkyl, or C₂ to C₆ alkoxyalkyl;

wherein each R^{b} and R^{d} are independently —H or optionally substituted aryl, aralkyl, heteroaryl, heteroaralkyl, or C₁ to C₄ alkyl, and each R^{c} is independently —H or optionally substituted C₁ to C₄ alkyl, aryl, or aralkyl, or NR^{c}₂ is an optionally substituted nonaromatic heterocycle.

In another embodiment, at least two of R₁, R₂, R₃ and R₄ are —H.

In a particular embodiment, each R^{b}, R^{c}, and R^{d} are, independently, —H, or optionally substituted C₁ to C₄ alkyl or phenyl, or each NR^{c}₂ is an optionally substituted morpholinyl, piperidyl, or piperazyl. Preferably, each R^{b}, R^{c}, and R^{d} is independently —H or C₁ to C₄ alkyl; or NR^{c}₂ is a nonaromatic heterocycle.

More preferably Formula I-a, one or two of R1 to R4 are each independently halogen, —(CO)R^{b}, —(CO)OR^{b}, —(CO)NR^{c}₂, —NR^{c}₂, —NR^{d}(CO)R^{b}, —NR^{d}(CO)OR^{b}, —NR^{d}(CO)NR^{c}₂, —NR^{d}(CO)PhNR^{d}(CO)R^{b}, or optionally substituted phenyl, benzyl, pyridyl, methylpyridyl, or optionally halogenated C₁ to C₄ alkyl or C₁ to C₄ alkoxy. In another preferable embodiment of Formula I-a, R₁, R₂, R₃, and R₄ are each, independently, —H, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C₃ to C₇ cycloalkyl, or nonaromatic heterocycle. More preferably, one or two of R₁ to R₄ are each independently —(CO)R^{b}, —(CO)OR^{b}, —(CO)NR^{c}₂, —NR^{c}₂, —NR^{d}(CO)R^{b}, —NR^{d}(CO)OR^{b}, —NR^{d}(CO)NR^{c}₂, —NR^{d}(CO)PhNR^{d}(CO)R^{b}, or optionally substituted phenyl, benzyl, pyridyl, or methylpyridyl.

In other preferred embodiments of Formula I-a at least one of R₁ to R₄ is —H, (CO)OR^{b}, e.g., —CO₂H or a C₁-C₄ carboxylic ester thereof. More typically, at least one of R₁ to R₄ is —CO₂H, or preferably, one of R₁ to R₃ is —CO₂H.

In other preferred embodiments of I-a, R₁, R₂, R₃ and R₄ are independently —HCOOR, —COR, —OC(O)R, —CONHR, —NHC(O)R, —COSR, —SC(O)R, —CH═CR, or —CH₂CHR, wherein R is defined as biphenyl, substituted biphenyl, substituted aryl, or heteroaryl.

In yet another preferred embodiment of I-a, R₁, R₂, and R₃ are each a hydrogen and ring A is a cyclopentyl or a cyclopentenyl ring.

In another preferred embodiment of I-a, R₄ is —COOR or —CONHR, wherein R is defined as biphenyl, substituted biphenyl, substituted aryl, or heteroaryl.

In a more preferred embodiment of I-a, R₄ is —COOR or —CONHR, wherein R is defined as biphenyl, substituted biphenyl, substituted aryl, or heteroaryl; wherein R is optionally substituted with —COOH, —P(O)₃Me₂ or tetrazole.

In an exemplary embodiment, the compound of Formula I-a is the compound of formula 4:

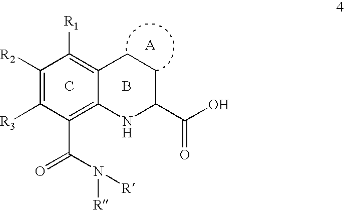

wherein R₁, R₂, R₃, A, B and C are as previously defined and R′ and R″ are each, independently, hydrogen, alkyl, substituted or unsubstituted aryl or heterocyclical.

In another exemplary embodiment, the compound of formula I-a is of the formula 5:

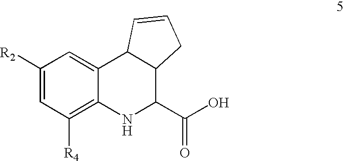

wherein R₂ is hydrogen, alkyl, alkoxyl or halogen and R₄ is a —CO₂aryl, —CH₂aryl or —OCH₂aryl group, wherein the aryl groups can be independently substituted with one or more groups selected from halogen, nitro, alkyl, cyano, hydroxyl, alkoxyl, amino or amido.

In one embodiment of the compound of formula I-a, R₁, R₂ and R₃ are each, independently, hydrogen or halogen; Ring A is cyclopentenyl; and R₄ is a —CO₂aryl, —CH₂aryl or —OCH₂aryl group, wherein the aryl groups can be optionally, independently, substituted one or more times with phenyl, halogen, nitro, alkyl, cyano, hydroxyl, alkoxyl, amino or amido.

In another embodiment of formula I-a, R₁, R₂ and R₃ are each hydrogen or halogen; R₄ is —CO₂phenyl, wherein the phenyl group can be independently substituted one or more times with halogen or phenyl; and A is cyclopentenyl.

In other embodiments of the compound, the compound of the method, and the compound of the pharmaceutical composition are each represented by the individual compounds provided in Table 1.

TABLE 1
Substituted Tetrahydroquinolines

| Com- | |
|---|---|
| pound # | Structure |
| 1 | 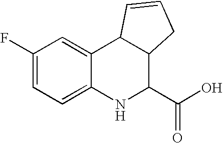 |
| 2 | 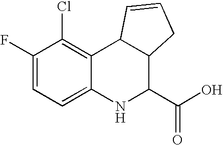 |
| 3 | 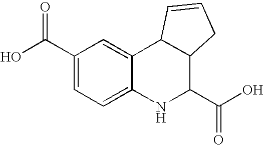 |
| 4 | 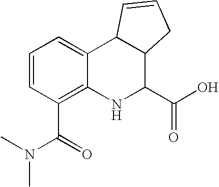 |
| 5 | 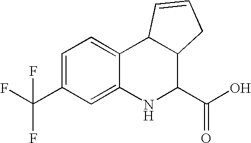 |
| 6 | 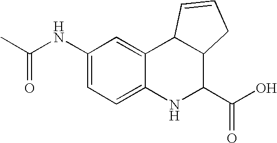 |
| 7 | 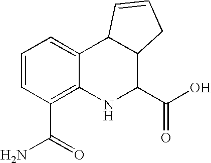 |
| 8 | 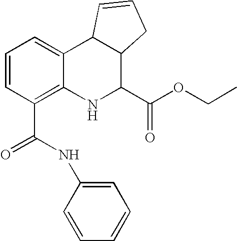 |
| 9 | 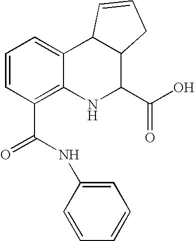 |
| 10 | 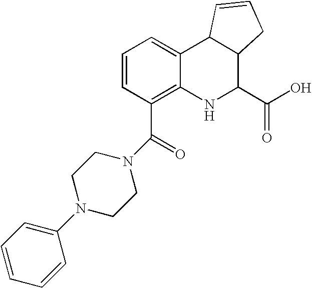 |
| 11 | 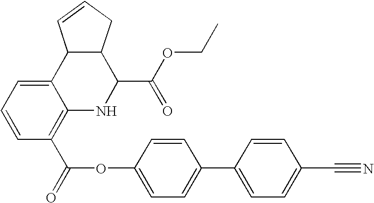 |
| 12 | 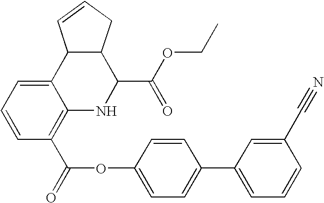 |
| 13 | 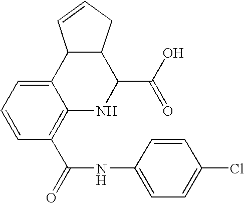 |
| 14 | 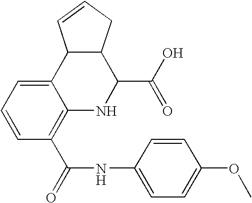 |
| 15 | 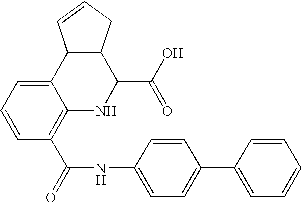 |
| 16 | 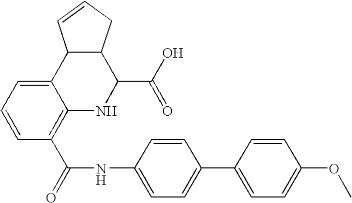 |
| 17 | 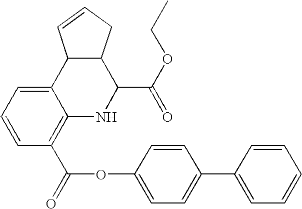 |
| 18 | 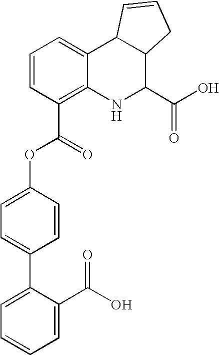 |
| 19 | 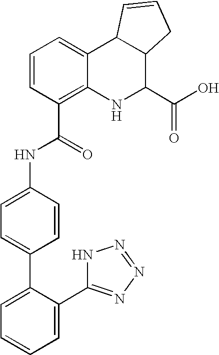 |
| 20 | 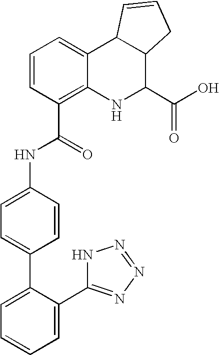 |
| 21 | 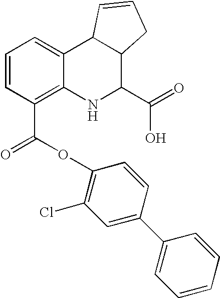 |
| 22 | 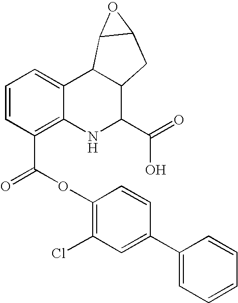 |
| 23 | 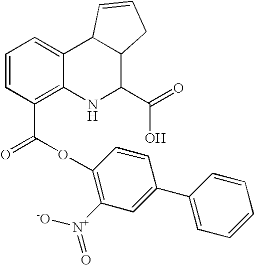 |
| 24 | 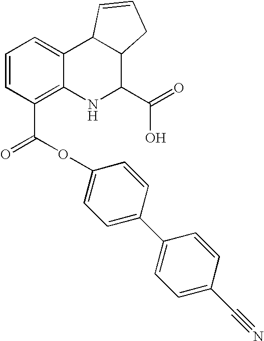 |
| 25 | 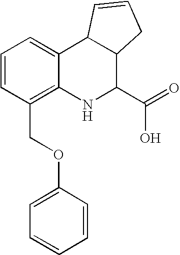 |
| 26 | 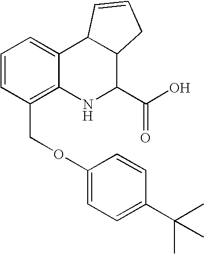 |
| 27 | 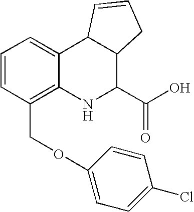 |
| 28 | 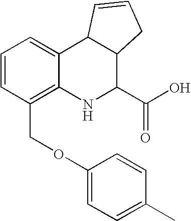 |
| 29 | 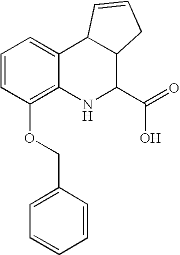 |
| 30 | 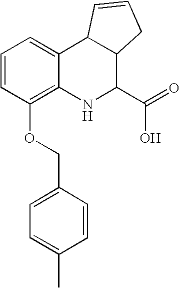 |
| 31 | 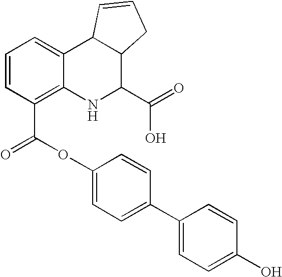 |
| 32 | 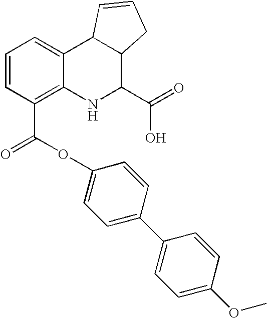 |
| 33 | 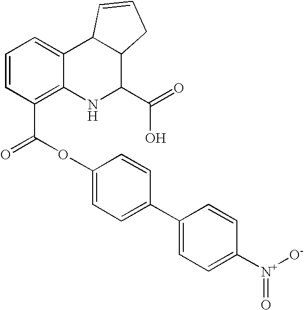 |
| 34 | 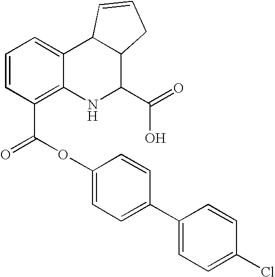 |
| 35 | 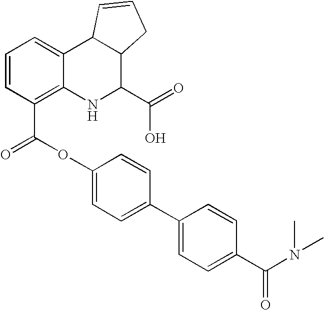 |
| 36 | 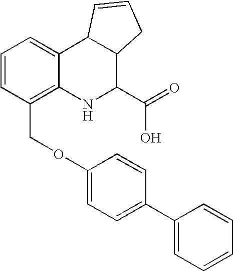 |
| 37 | 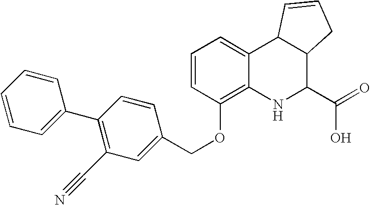 |
| 38 | 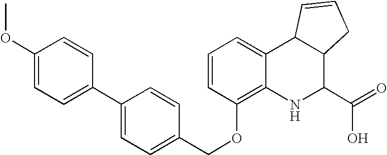 |
| 39 | 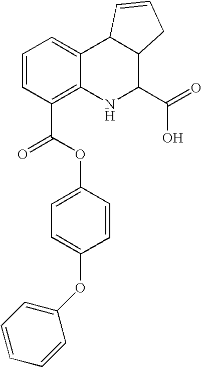 |
| 40 | 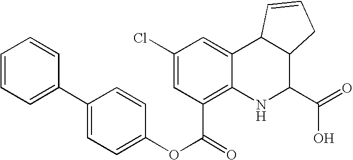 |
| 41 | 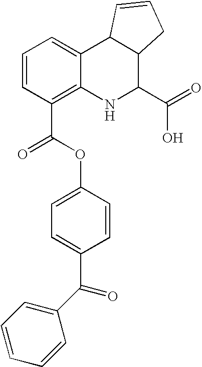 |
| 42 | 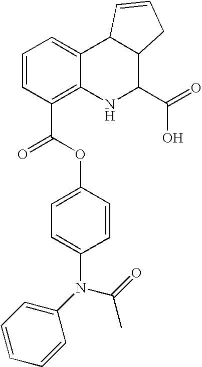 |
| 43 | 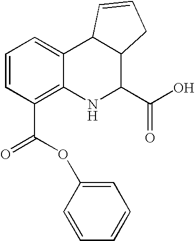 |
| 44 | 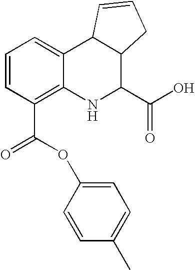 |
| 45 | 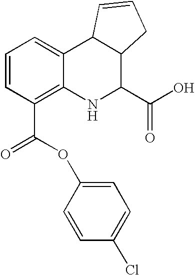 |
| 46 | 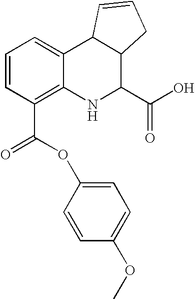 |
| 47 | 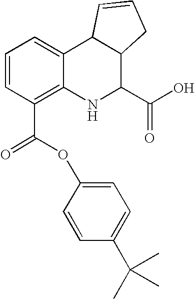 |
| 48 | 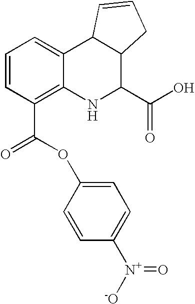 |
| 49 | 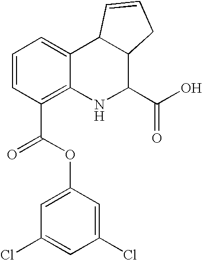 |
| 50 | 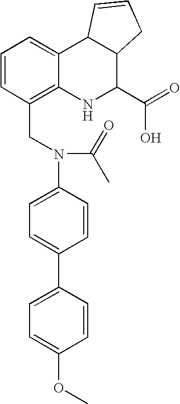 |

In a particular embodiment, the compounds of Table 1 can be used for the treatment of a bacterial infection in a subject in need thereof.

Compounds of the invention may be useful in the treatment of bacterial infections dependent on MurA. Thus, as a further embodiment, the present invention provides the use of a compound of formula I-a in therapy. In a further embodiment, the therapy is selected from a disease which is ameliorated by modulation of MurA.

It should be understood that any of the compounds depicted can be used to treat a subject in need of treatment for a bacterial infection by administering an effective amount of any compound depicted.

It should be understood that any of the compounds depicted can be used to treat an infection caused by a bacterium that expresses phosphoenolpyruvate:UDP-N-acetyl-D-glucosamine 1-carboxyvinyltransferase.

In a preferred embodiment, the method of identifying compounds as MurA inhibitors is combined with one or more assays for antibiotic activity. Such assays are well known in the art, and can include, for example, contacting bacteria of interest with a test compound under conditions otherwise suitable for bacterial growth, and determining if the test compound has antibacterial activity.

Also included in the present invention are pharmaceutical compositions comprising the disclosed MurA inhibitors, (e.g., I-a). The present invention also includes novel MurA inhibitors disclosed herein (e.g., I-a), or pharmaceutically acceptable, salts, solvates or hydrates thereof.

A “subject” includes mammals, e.g., humans, companion animals (e.g., dogs, cats, birds, aquarium fish, reptiles, and the like), farm animals (e.g., cows, sheep, pigs, horses, fowl, farm-raised fish and the like) and laboratory animals (e.g., rats, mice, guinea pigs, birds, aquarium fish, reptiles, and the like). Alternatively, the subject is a warm-blooded animal. More preferably, the subject is a mammal. Most preferably, the subject is human.

A subject in need of treatment has a bacterial infection (or has been exposed to an infectious environment where bacteria are present, e.g., in a hospital) the symptoms of which may be alleviated by administering an effective amount of the disclosed MurA inhibitors. For example, a subject in need of treatment can have an infection for which the disclosed MurA inhibitors can be administered as a treatment. In another example, a subject in need of treatment can have an open wound or burn injury, or can have a compromised immune system, for which the disclosed MurA inhibitors can be administered as a prophylactic. Thus, a subject can be treated therapeutically or prophylactically. More preferably, a subject is treated therapeutically.

Typically, the subject is treated for a bacterial infection caused by a bacteria of a genus selected from Allochromatium, Acinetobacter, Bacillus, Campylobacter, Chlamydia, Chlamydophila, Clostridium, Citrobacter, Escherichia, Enterobacter, Enterococcus, Francisella, Haemophilus, Helicobacter, Klebsiella, Listeria, Moraxella, Mycobacterium, Neisseria, Proteus, Pseudomonas, Salmonella, Serratia, Shigella, Stenotrophomonas, Staphyloccocus, Streptococcus, Synechococcus, Vibrio, and Yersina.

More preferably, the subject is treated for a bacterial infection from Allochromatium vinosum, Acinetobacter baumanii, Bacillus anthracis, Campylobacter jejuni, Chlamydia trachomatis, Chlamydia pneumoniae, Clostridium spp., Citrobacter spp., Escherichia coli, Enterobacter spp., Enterococcus faecalis., Enterococcus faecium, Francisella tularensis, Haemophilus influenzae, Helicobacter pylori, Klebsiella spp., Listeria monocytogenes, Moraxella catharralis, Mycobacterium tuberculosis, Neisseria meningitidis, Neisseria gonorrhoeae, Proteus mirabilis, Proteus vulgaris, Pseudomonas aeruginosa, Salmonella spp., Serratia spp., Shigella spp., Stenotrophomonas maltophilia, Staphyloccocus aureus, Staphyloccocus epidermidis, Streptococcus pneunmoniae, Streptococcus pyogenes, Streptococcus agalactiae, Yersina pestis, and Yersina enterocolitica, and the like.

Preferably, the subject is treated for a bacterial infection caused by a bacterium that expresses a peptidoglycan biosynthesis pathway, and in particular, expresses the enzyme encoded by the MurA/MurZ gene. Numerous studies have demonstrated that the MurA gene and its paralog MurZ are conserved across a range of Gram positive and Gram negative bacteria; see, for example, Schonbrunn E, Eschenburg S, Krekel F, Luger K, Amrhein N. (2000) Biochemistry. 2000 Mar. 7; 39(9):2164-73; Baum E Z, Montenegro D A, Licata L, Turchi I, Webb G C, Foleno B D, Bush K. (2001)Antimicrob Agents Chemother. 2001 November; 45(11):3182-8; Kim D H, Lees W J, Kempsell K E, Lane W S, Duncan K, Walsh C T. (1996) Biochemistry. 1996 Apr. 16; 35(15):4923-8; and Skarzynski T, Mistry A, Wonacott A, Hutchinson S E, Kelly V A, Duncan K. (1996) Structure. 1996 Dec. 15; 4(12):1465-74. The entire teachings of these documents are incorporated herein by reference.

As used herein, the term MurA, referring to the gene or the enzyme thereby encoded, encompasses both MurA and its paralog MurZ. The term MurA represents both the gene murA and the enzyme MurA. The enzymes are given various names in the art, including, for example: MurA transferase; MurZ transferase; UDP-N-acetylglucosamine 1-carboxyvinyl-transferase; UDP-N-acetylglucosamine enoylpyruvyl transferase; UDP-N-acetyl glucosamine enolpyruvyltransferase; enoylpyruvate transferase; phosphoenolpyruvate-UDP-acetylglucosamine-3-enolpyruvyltransferase; phosphoenolpyruvate:UDP-2-acetamido-2-deoxy-D-glucose 2-enoyl-1-carboxyethyltransferase; phosphoenolpyruvate:uridine diphosphate N-acetyl glucosamine enolpyruvyltransferase; phosphoenolpyruvate:uridine-5′-diphospho-N-acetyl-2-amino-2-deoxyglucose 3-enolpyruvyltransferase; phosphopyruvate-uridine diphosphoacetylglucosamine pyruvatetransferase; pyruvate-UDP-acetyl glucosamine transferase; pyruvate-uridine diphospho-N-acetyl glucosamine transferase; pyruvate-uridine diphospho-N-acetyl-glucosamine transferase; or pyruvic-uridine diphospho-N-acetylglucosaminyltransferase.

As used herein, the term MurB, referring to the gene or the enzyme thereby encoded, is given various names in the art, including, for example: UDP-N-acetylmuramate dehydrogenase, MurB reductase; UDP-N-acetylenol pyruvoyl glucosamine reductase; UDP-N-acetylglucosamine-enoylpyruvate reductase; UDP-GlcNAc-enoylpyruvate reductase; uridine diphosphoacetylpyruvoylglucosamine reductase; uridine diphospho-N-acetylglucosamine-enolpyruvate reductase; uridine-5′-diphospho-N-acetyl-2-amino-2-deoxy-3-O-lactylglucose:NADP-oxidoreduct-ase.

The systematic name typically given for MurA/MurZ is phosphoenolpyruvate: UDP-N-acetyl-D-glucosamine 1-carboxyvinyltransferase, and the IUBMB systematic classification is EC 2.5.1.7. The systematic name typically given for MurB is UDP-N-acetylmuramate:NADP⁺oxidoreductase, and the IUBMB systematic classification is EC 1.1.1.158. See International Union of Biochemistry and Molecular Biology online at www.chem.qmul.ac.uk/iubmb/.

In other embodiments, bacterial growth can be retarded, modulated, or prevented by employing an effective amount of the disclosed MurA inhibitors. Numerous bacteria can express the MurA enzyme. Bacteria that express MurA can include, for example, actinobacteria, bacteroids, chlamydia, cyanobacteria; firmicutes, e.g., bacillales, clostridia, and lactobacillales; fusobacteria; green sulfur bacteria; hyperthermophilic bacteria; proteobacteria, e.g., alpha, beta, delta, epsilon, and gamma; radioresistant bacteria; and spirochetes.

For example, actinobacteria can include, Bifidobacterium longum, Corynebacterium efficiens, Corynebacterium glutamicum, Mycobacterium bovis, Mycobacterium leprae, Mycobacterium tuberculosis (e.g., CDC1551 and H37Rv (lab strain)), Streptomyces coelicolor, Tropheryma whipplei (e.g., Twist, TW08/27); and the like.

Examples of bacteroids include Bacteroides thetaiotaomicron and the like.

Chlamydia can include, e.g., Chlamydophila caviae, Chlamydia muridarum, Chlamydophila pneumoniae (e.g., AR39, J138, CWL029, Chlamydia trachomatis) and the like.

Examples of cyanobacteria can include Anabaena sp. PCC7120 (Nostoc sp. PCC7120), Synechocystis sp. PCC6803, Thermosynechococcus elongates, and the like.

Firmicutes, e.g., bacillales can include Bacillus cereus, Bacillus halodurans, Bacillus subtilis, Listeria innocua, Listeria monocytogenes, Oceanobacillus iheyensis, Staphylococcus aureus (e.g., MW2, N315, and Mu50), Staphylococcus epidermidis, and the like.

Firmicutes, e.g., clostridia, can include Clostridium acetobutylicum, Clostridium perfringens, Clostridium tetani, Thermoanaerobacter tengcongensis, and the like.

Firmicutes, e.g., lactobacillales, can include Enterococcus faecalis, Lactococcus lactis, Lactobacillus plantarum, Streptococcus agalactiae (e.g., 2603 and NEM316), Streptococcus mutans, Streptococcus pyogenes (e.g., MGAS315 (serotype M3), SF370 (serotype M1), SSI-1 (serotype M3), and MGAS8232 (serotype M18)), Streptococcus pneumoniae (e.g., TIGR4 and R6), and the like.

Fusobacteria can include Fusobacterium nucleatum, and the like.

Green sulfur bacteria can include Chlorobium tepidum, and the like.

Hyperthermophilic bacteria can include Aquifex aeolicus, Thermotoga maritime, and the like.

Examples of alpha proteobacteria can include Agrobacterium tumefaciens C58 (Cereon), Bradyrhizobium japonicum, Brucella melitensis, Brucella suis, Caulobacter crescentus, Mesorhizobium loti, Rickettsia conorii, Rickettsia prowazekii, Sinorhizobium meliloti, and the like.

Examples of beta proteobacteria can include Nitrosomonas europaea, Neisseria meningitidis (e.g., Z2491 (serogroup A) and MC58 (setogroup B), Ralstonia solanacearum, and the like.

Examples of delta/epsilon proteobacteria can include Campylobacter jejuni, Helicobacter pylori (e.g., J99 and 26695), and the like.

Examples of gamma proteobacteria can include Buchnera aphidicola (e.g., Baizongia pistaciae), Buchnera aphidicola (e.g., Schizaphis graminum), Buchnera sp. APS (e.g., Acyrthosiphon pisum), Coxiella burnetii, Escherichia coli (e.g., CFT073, O157 EDL933, K-12 W3110, K-12 MG1655, and O157 Sakai), Haemophilus influenzae, Pseudomonas aeruginosa, Pasteurella multocida, Pseudomonas putida, Pseudomonas syringae pv., Shigella flexneri 301 (serotype 2a), Shewanella oneidensis, Salmonella typhimurium, Salmonella typhi (e.g., Ty2, CT18), Vibrio cholerae, Vibrio parahaemolyticus, Vibrio vulnificus, Wigglesworthia brevipalpis, Xanthomonas axonopodis, Xanthomonas campestris, Xylella fastidiosa (e.g., 9a5c and Temecula1), Yersinia pestis (e.g., CO92 and KIM), and the like.

Radioresistant bacteria can include Deinococcus radiodurans, and the like.

Spirochetes can include Borrelia burgdorferi, Leptospira interrogans, Treponema pallidum, and the like.

In one embodiment, a subject is also concurrently treated for a fungal infection, for example, a fungal infection caused by a pathogenic dermatophyte, e.g., a species of the genera Trichophyton, Tinea, Microsporum, Epidermophyton and the like; or a pathogenic filamentous fungus, e.g., a species of genera such as Aspergillus, Histoplasma, Cryptococcus, Microsporum, and the like; or a pathogenic non-filamentous fungus, e.g., a yeast, for example, a species of the genera Candida, Malassezia, Trichosporon, Rhodotorula, Torulopsis, Blastomyces, Paracoccidioides, Coccidioides, and the like. Preferably, the subject is concurrently treated for a fungal infection resulting from a species of the genera Aspergillus or Trichophyton. Species of Trichophyton include, for example, T. mentagrophytes, T. rubrum, T. schoenleinii, T. tonsurans, T. verrucosum, and T. violaceum. Species of Aspergillus include, for example, A. fumigatus, A. flavus, A. niger, A. amstelodami A. candidus, A. carneus, A. nidulans, A oryzae, A. restrictus, A. sydowi, A. terreus, A. ustus, A. versicolor, A. caesiellus, A. clavatus, A. avenaceus, and A. deflectus. More preferably, the subject is concurrently treated therapeutically for a fungal infection caused by a species of the genus Aspergillus selected from A. fumigatus, A. flavus, A. niger, A. amstelodami, A. candidus, A. carneus, A. nidulans, A oryzae, A. restrictus, A. sydowi, A. terreus, A. ustus, A. versicolor, A. caesiellus, A. clavatus, A. avenaceus, and A. deflectus. Even more preferably the subject is concurrently treated therapeutically for a fungal infection caused by Aspergillus fumigatus or Aspergillus niger, and most preferably, Aspergillus fumigatus.

An “effective amount” of a compound of the disclosed invention is the quantity that, when administered to a subject in need of treatment, improves the prognosis of the subject, e.g., delays the onset of and/or reduces the severity of one or more of the subject's symptoms associated with a bacterial infection. The amount of the disclosed compound to be administered to a subject will depend on the particular disease, the mode of administration, co-administered compounds, if any, and the characteristics of the subject, such as general health, other diseases, age, sex, genotype, body weight and tolerance to drugs. The skilled artisan will be able to determine appropriate dosages depending on these and other factors. Effective amounts of the disclosed compounds typically range between about 0.01 mg/kg per day and about 100 mg/kg per day, and preferably between 0.1 mg/kg per day and about 10 mg/kg/day. Techniques for administration of the disclosed compounds of the invention can be found in Remington: the Science and Practice of Pharmacy, 19^{th} edition, Mack Publishing Co., Easton, Pa. (1995), the entire teachings of which are incorporated herein by reference.

A “pharmaceutically acceptable salt” of the disclosed compound is a product of the disclosed compound that contains an ionic bond, and is typically produced by reacting the disclosed compound with either an acid or a base, suitable for administering to a subject.

For example, an acid salt of a compound containing an amine or other basic group can be obtained by reacting the compound with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid.

Salts of compounds containing a carboxylic acid or other acidic functional groups can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl-beta-phenethylamine, dehydroabietylamine, N,N′-bisdehydroabietylamine, glucamine, N-methylglucamine, collidine, quinine, quinoline, and basic amino acid such as lysine and arginine.

Certain compounds and their salts may also exist in the form of solvates, for example hydrates, and the present invention includes each solvate and mixtures thereof.

As used herein, a “pharmaceutical composition” is a formulation containing the disclosed compounds in a form suitable for administration to a subject. The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of active ingredient (i.e., a formulation of the disclosed compound or salts thereof) in a unit dose of composition is an effective amount and may be varied according to the particular treatment involved. It is appreciated that it may be necessary to make routine variations to the dosage depending on the age and condition of the patient. The dosage will also depend on the route of administration. A variety of routes are contemplated, including topical, oral, pulmonary, rectal, vaginal, parenternal, transdermal, subcutaneous, intravenous, intramuscular, intraperitoneal and intranasal.

The compounds described herein, and the pharmaceutically acceptable salts thereof, can be used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein. Techniques for formulation and administration of the disclosed compounds of the invention can be found in Remington: the Science and Practice of Pharmacy, above.

For oral administration, the disclosed compounds or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like.

For parental administration of the disclosed compounds, or salts, solvates, or hydrates thereof, can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations previously described, the compounds may also be formulated as a depot preparation. Suitable formulations of this type include biocompatible and biodegradable polymeric hydrogel formulations using crosslinked or water insoluble polysaccharide formulations, polymerizable polyethylene oxide formulations, impregnated membranes, and the like. Such long acting formulations may be administered by implantation or transcutaneous delivery (for example subcutaneously or intramuscularly), intramuscular injection or a transdermal patch. Preferably, they are implanted in, or applied to, the microenvironment of an affected organ or tissue, for example, a membrane impregnated with the disclosed compound can be applied to an open wound or burn injury. Thus, for example, the compounds may be formulated with suitable polymeric or hydrophobic materials, for example, as an emulsion in a acceptable oil, or ion exchange resins, or as sparingly soluble derivatives, for example, as a sparingly soluble salt.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, a cream formulation can be administer to a wound site, or a bandage may be impregnated with a formulation, and the like.

For rectal administration, suitable pharmaceutical compositions are, for example, topical preparations, suppositories or enemas.

For vaginal administration, suitable pharmaceutical compositions are, for example, topical preparations, pessaries, tampons, creams, gels, pastes, foams or sprays.

In addition, the compounds may also be formulated to deliver the active agent by pulmonary administration, e.g., administration of an aerosol formulation containing the active agent from, for example, a manual pump spray, nebulizer or pressurized metered-dose inhaler. Suitable formulations of this type can also include other agents, such as antistatic agents, to maintain the disclosed compounds as effective aerosols.

The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

For nasal administration, either a solid or a liquid carrier can be used. The solid carrier includes a coarse powder having particle size in the range of, for example, from about 20 to about 500 microns and such formulation is administered by rapid inhalation through the nasal passages. Where the liquid carrier is used, the formulation may be administered as a nasal spray or drops and may include oil or aqueous solutions of the active ingredients.

In addition to the formulations described above, a formulation can optionally include, or be co-administered with one or more additional drugs, e.g., other antibiotics, anti-inflammatories, antifungals, antivirals, immunomodulators, antiprotozoals, steroids, decongestants, bronchodialators, and the like. For example, the disclosed compound can be co-administered with drugs such as such as ibuprofen, prednisone (corticosteroid) pentoxifylline, Amphotericin B, Fluconazole, Ketoconazol, Itraconazol, penicillin, ampicillin, amoxicillin, and the like. The formulation may also contain preserving agents, solubilizing agents, chemical buffers, surfactants, emulsifiers, colorants, odorants and sweeteners.

The term “aryl” group, refers to carbocyclic aromatic groups such as phenyl, naphthyl, tetrahydronaphthyl, anthracyl, and the like. The term “heteroaryl” group refers to heteroaromatic groups, for example imidazolyl, isoimidazolyl, thienyl, furanyl, pyridyl, pyrimidyl, pyranyl, pyrazolyl, pyrrolyl, pyrazinyl, thiazolyl, isothiazolyl, oxazolyl, isooxazolyl, 1,2,3-triazolyl, 1,2,4-triazolyl, tetrazolyl, benzo[1,3]dioxolyl, 2,3-dihydro-benzo[1,4]dioxine, benzopyrimidyl, benzopyrazyl, benzofuranyl, indolyl, benzothienyl, benzoxazolyl, benzoisooxazolyl, benzothiazolyl, benzoisothiazolyl, quinolinyl, isoquinolinyl, benzimidazolyl, tetrahydroquinolinyl, and tetrahydroisoquinolinyl. Preferable aryl and heteroaryl groups include phenyl and pyridyl. The term “Ph” indicates a phenyl or a phenylene group.

The term “nonaromatic heterocycle” refers to non-aromatic ring systems typically having three to eight members, preferably five to six, in which one or more ring carbons, preferably one to four, are each replaced by a heteroatom such as N, O, or S. Examples of non-aromatic heterocyclic rings include 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothienyl, 3-tetrahydrothienyl, N-morpholinyl, 2-morpholinyl, 3-morpholinyl, 4-morpholinyl, N-thiomorpholinyl, 2-thiomorpholinyl, 3-thiomorpholinyl, 4-thiomorpholinyl, 1-pyrrolidyl, 2-pyrrolidyl, 3-pyrorolidyl, 1-piperazyl, 2-piperazyl, 1-piperidyl, 2-piperidyl, 3-piperidyl, 4-piperidyl, 4-thiazolidyl, diazolonyl, N-substituted diazolonyl, 1-pthalimidyl, azetidyl, aziridyl, oxaziridyl, oxazolidyl, isooxazolidyl, thiazolidyl, isothiazolidyl, oxazinanyl, thiazinanyl, azepanyl, oxazepanyl, and thiazepanyl. Typically, the nonaromatic heterocycle groups represented by NR^{c}₂ and NR^{j}₂ are selected from optionally substituted pyrrolidyl, piperidyl, piperazyl, morpholinyl, and thiomorpholinyl, or preferably, unsubstituted piperidyl or morpholinyl.

The disclosed compounds can contain one or more chiral centers. The presence of chiral centers in a molecule gives rise to stereoisomers. For example, a pair of optical isomers, referred to as “enantiomers,” exist for every chiral center in a molecule. A pair of diastereomers exists for every chiral center in a compound having two or more chiral centers. Where the structural formulas do not explicitly depict the stereochemistry of each chiral center, for example in structural formula I-a, it is to be understood that these formulas encompass enantiomers free from the corresponding optical isomer, racemic mixtures, mixtures enriched in one enantiomer relative to its corresponding optical isomer, a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s).

The term “alkyl” used alone or as part of a larger moiety (e.g., aralkyl, alkoxy, alkylamino, alkylaminocarbonyl, haloalkyl), is a straight or branched non-aromatic hydrocarbon which is completely saturated. Typically, a straight or branched alkyl group has from 1 to about 10 carbon atoms, preferably from 1 to about 5 if not otherwise specified. Examples of suitable straight or branched alkyl group include methyl, ethyl, n-propyl, 2-propyl, n-butyl, sec-butyl, tert-butyl, pentyl, hexyl, heptyl or octyl. A C₁ to C₁₀ straight or branched alkyl group or a C₃ to C₈ cyclic alkyl group can also be referred to as a “lower alkyl” group. An “alkoxy” group refers to an alkyl group that is connected through an intervening oxygen atom, e.g., methoxy, ethoxy, 2-propyloxy, tert-butoxy, 2-butyloxy, 3-pentyloxy, and the like.

The terms “optionally halogenated alkyl,” and “optionally halogenated alkoxy,” as used herein, includes the respective group substituted with one or more of —F, —Cl, —Br, or —I.

The terms “alkanoyl,” “aroyl,” and the like, as used herein, indicates the respective group connected through an intervening carbonyl, for example, —(CO)CH₂CH₃, benzoyl, and the like. The terms “alkanoyloxy,” “aroyloxy,” and the like, as used herein, indicates the respective group connected through an intervening carboxylate, for example, —O(CO)CH₂CH₃, —O(CO)C₆H₅, and the like.

The term “cycloalkyl group” is a cyclic alkyl group having from 3 to about 10 carbon atoms, preferably from 5 to 6. Examples of suitable cycloalkyl groups include cyclopropyl, cyclobutyl, cyclopentyl, cyclohexyl, cycloheptyl and cyclooctyl. A “cycloalkoxy” group refers to a cycloalkyl group that is connected through an intervening oxygen atom, e.g., cyclopentyloxy, cyclohexyloxy, and the like.

The term “cycloalkenyl” includes nonaromatic cycloalkyl groups that contain one or more units of carbon-carbon unsaturation, i.e., carbon-carbon double bonds. A cycloalkenyl group includes, for example, cyclohexenyl or cyclopentenyl.

The terms “aralkyl,” “heteroaralkyl,” “cycloalkylalkyl,” and “nonaromatic heterocycloalkyl” refer to aryl, heteroaryl, cycloalkyl, and nonaromatic heterocycle groups, respectively, that are connected through an alkyl chain, e.g., benzyl, —CH₂CH₂-pyridine, (3-cyclohexyl)propyl, and the like.

An “acyclic” group is a substituent that does not contain a ring. A “monocyclic” group contains only a single ring, for example, a phenyl ring that is not fused to another ring. A “polycyclic” group is a group that contains multiple fused rings, for example, naphthyl.

The term “derivative,” e.g., in the term “substituted tetrahydroquinoline derivatives,” refers to compounds that have a common core structure, and are substituted with various groups as described herein. For example, all of the compounds represented by formula Ia are substituted tetrahydroquinoline derivatives.

A line across a bond in a ring indicates that the represented bond can be connected to any substitutable atom in the ring.

A “substitutable atom” is any atom such as nitrogen or carbon that can be substituted by replacing a hydrogen atom bound to the atom with a substituent. A “substitutable ring atom” in a ring, e.g., the substitutable ring carbons in Rings A to C, is any ring atom, e.g., a carbon or nitrogen, which can be substituted.

Suitable substituents are those that do not substantially interfere with the pharmaceutical activity of the disclosed compound. A compound or group can have one or more substituents, which can be identical or different. Examples of suitable substituents for a substitutable carbon atom in an alkyl, cycloalkyl, cycloalkenyl, non-aromatic heterocycle, aryl, or heteroaryl group include —OH, halogen (—Br, —Cl, —I and —F), —R, —OR, —CH₂R, —CH₂CH₂R, —OCH₂R, —CH₂OR, —CH₂CH₂OR, —CH₂OC(O)R, —O—COR, —COR, —SR, —SCH₂R, —CH₂SR, —SOR, —SO₂R, —CN, —NO₂, —COOH, —SO₃H, —NH₂, —NHR, —N(R)₂, —COOR, —CH₂COOR, —CH₂CH₂COOR, —CHO, —CONH₂, —CONHR, —CON(R)₂, —NHCOR, —NRCOR, —NHCONH₂, —NHCONRH, —NHCON(R)₂, —NRCONH₂, —NRCONRH, —NRCON(R)₂, —C(═NH)—NH₂, —C(═NH)—NHR, —C(═NH)—N(R)₂, —C(═NR)—NH₂, —C(═NR)—NHR, —C(═NR)—N(R)₂, —NH—C(═NH)—NH₂, —NH—C(═NR)—NHR, —NH—C(═NH)—N(R₂, —NH—C(═NR)—NH₂, —NH—C(═NR)—NHR, —NH—C(═NR)—N(R)₂, —NRH—C(═NH)—NH₂, —NR—C(═NH)—NHR, —NR—C(═NH)—N(R)₂, —NR—C(═NR)—NH₂, —NR—C(═NR)—NHR, —NR—C(═NR)—N(R)₂, —SO₂NH₂, —SO2NHR, —SO₂NR₂, —SH, —SOₖR (k is 0, 1 or 2) and —NH—C(═NH)—NH₂. Each R is independently an alkyl, cycloalkyl, benzyl, aromatic, heteroaromatic, or phenylamine group that is optionally substituted. Preferably, R is unsubstituted. In addition, —N(R)₂, taken together, can also form a substituted or unsubstituted heterocyclic group, such as pyrrolidinyl, piperidinyl, morpholinyl and thiomorpholinyl. Examples of substituents on group represented by R include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl.

Suitable substituents on the nitrogen of a heterocyclic group or heteroaromatic group include —R′, —N(R′)₂, —C(O)R′, —CO₂R, —C(O)C(O)R′, —C(O)CH₂C(O)R′, —SO₂R′, —SO₂N(R′)₂, —C(═S)N(R′)₂, —C(═NH)—N(R′)₂, and —NR′SO₂R′. R′ is hydrogen, an alkyl, alkoxy, cycloalkyl, cycloalkoxy, phenyl, phenoxy, benzyl, benzyloxy, heteroaromatic, or heterocyclic group that is optionally substituted. Examples of substituents on the groups represented by R′ include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl. Preferably, R′ is unsubstituted.

Advantageously, the present invention also provides kits for use by a consumer for treating disease. The kits comprise a) a pharmaceutical composition comprising the substituted tetrahydraquinoline antibacterial agent and a pharmaceutically acceptable carrier, vehicle or diluent; and, optionally, b) instructions describing a method of using the pharmaceutical composition for treating the specific disease. The instructions may also indicate that the kit is for treating disease while substantially reducing the concomitant liability of adverse effects associated with antibiotic administration.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that, has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXEMPLIFICATION

### Example 1

### Synthesis of MurA Inhibitors of Structural Formula I-a

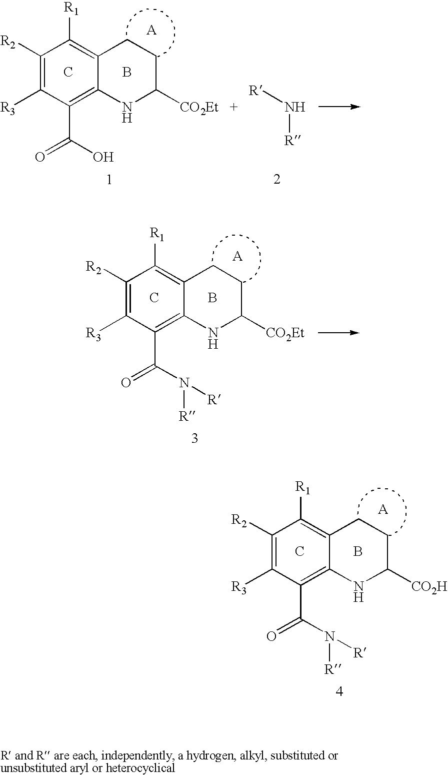

To a solution of 1 and aniline 2 in dry DMF is added HOBT and 1-methylmorpholine followed by EDCI at room temperature under N₂. The resulting mixture is stirred for 16 hours. Most of the solvent is removed in-vacuo and the residue is treated with 1N citric acid and CH₂Cl₂. The organic layer is separated and the aqueous layer is extracted with CH₂Cl₂. The combined organic layer is dried with Na₂SO₄ and the solvent is removed in vacuo. The product 3 can be further purified by silica gel chromatography. To a solution of 3 in THF—H₂O (9/1) was added LiOH—H₂O at room temperature and the resulting mixture is stirred for 20 hours. Most of THF is removed in-vacuo and the residue is treated with 1N Citric acid. This is extracted with CH₂Cl₂. The combined organic layer is dried with Na₂SO₄. The solvent was removed in-vacuo. The solid residue is recrystallized give compound 4 which is was dried in a vacuum oven at 60° C. overnight.

In order to make ester derivatives of the above compounds (e.g. compounds 21 and 40 from Table 1), aniline 2 can be replaced with an alcohol (e.g. Ph-OH, e.g., wherein the Ph can be substituted).

### Example 2

### Synthesis of MurA Inhibitor

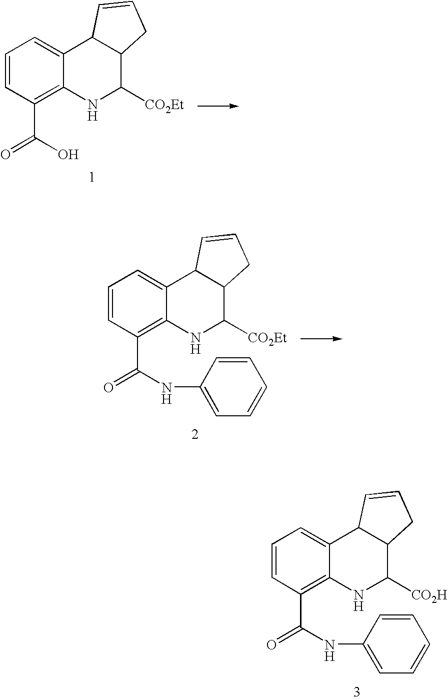

To a solution of 1 (205 mg, 0.71 mM) and aniline (72 mg, 0.77 mM) in dry DMF (20 mL) was added HOBT (1 mL, 0.5 M in DMF, 0.5 mM) and 1-methylmorpholine (152 mg, 1.5 mM) followed by EDCI (285 mg, 1.48 mM) at room temperature under N₂. The resulting mixture was stirred for 16 hours. Most of the solvent was removed in vacuo and the oily residue was treated with 1N citric acid (20 mL) and CH₂Cl₂ (50 mL). The organic layer was separated and the aqueous layer was extracted with CH₂Cl₂ (2×30 mL). The combined organic layer was dried with Na₂SO₄ and the solvent was removed in vacuo. The residue was purified by column chromatography on silica gel (ethyl acetate/hexane (1/4 to 1/3) to give compound 2 (210 mg, 81%) as an off-white powder. ¹H-NMR (DMSO-d₆): 1.22 (3H, CH₃CH₂O—), 2.28 (2H, CH₂—CH═CH), 3.21 (1H, CH—CHNHCO₂), 4.20 (4H, CH₃CH₂O, ArCH—CH═CH and NHCH—CO₂), 5.61 and 5.78 (1 H each, olefinic protons), 10.12 (1H, CONH—). MS:M+1=363.

To a solution of 2 (121 mg, 0.33 mM) in THF—H₂O (9/1) (10 mL) was added LiOH—H₂O (50 mg, 1.19 mM) at room temperature and the resulting mixture was stirred for 20 hours. Most of THF was removed in vacuo and the residue was treated with 1N citric acid (15 mL). This was extracted with CH₂Cl₂ (3×30 mL), the organic layers were combined and dried with Na₂SO₄ followed by removal of the solvent in vacuo. The solid residue was recrystallized from CHCl₃ and hexane to give compound 3. This was dried in vacuum oven at 60° C. to give compound 3 (96 mg, 85%) as an off-white powder. ¹H-NMR (DMSO-d₆): 2.28 (2H, CH₂—CH═CH), 3.21 (1H, CH—CHNHCO₂), 4.20 (2H, ArCH—CH═CH and NHCH—CO₂), 5.61 and 5.78 (1 H each, olefinic protons), 10.10 (1H, CONH—). MS:M+1=335.

### Example 3

### High Throughput Screen can Identify Likely MurA Inhibitors

A high throughput screen is employed on the compounds to identify MurA inhibitors. The test conditions employ MurA and MurB (UDP-N-acetylmuramate: NADP+oxidoreductase, EC 1.1.1.158) coupled enzymatic reactions carried in 96-well reaction plates.

Using appropriate stock solutions, each well is prepared to contain a total volume of about 100 μL, containing 50 mM Tris-HCl (Tris(hydroxymethyl)aminomethane-HCl, pH 8.0), 20 mM KCl, 0.02% Brij.RTM.30 (Polyethylene glycol dodecyl ether), 0.5 mM DTT (dithiothreitol), 0.1 mM UDPAG (Uridine 5′-diphospho-N-acetylglucosamine), 0.1 mM phosphoenolpyruvate (PEP), 0.1 mM NADPH (nicotinamide adenine dinucleotide phosphate), 120 ng MurA, and 40 ng MurB. The preceding chemical reagents are obtained from Sigma, St. Louis Mo.; the enzymes are produced in house.

The wells are prepared without substrate (PEP and UDPAG) incubated for a half hour, combined with the substrate and each test compound, and the evidence of reaction is read after 1 hour of reaction time using a fluorescence spectrometer at 355/460 nM for 0.1 second. Compounds associated with an increase in fluorescence over control solutions are identified as likely MurA inhibitors.

### Example 4

### Kinetic Assay of Disclosed Inhibitors Shows Potent MurA Inhibition

A series of IC₅₀ (Inhibition Concentration at 50 percent) assays are performed in 96-well assay plates. Compounds with IC₅₀ values>200 can have a measurable IC₅₀ using a different assay method. About 60 μL of a buffer A1 is added into each well from column 1 to column 12. An additional 20 μL of buffer A1 is added into column 12. Buffer A1 is prepared to contain 50 mM HEPES pH 7.5 (4-(2-hydroxyethyl)piperazine-1-ethanesulfonic acid), 20 mM KCl, 0.02% wt Brij 30, 0.001 mM UDPAG, 0.001 mM PEP, and 0.5 mM DTT.

TABLE 2
IC₅₀ data for Substituted Tetrahydroquinolines

| Compound # | EPT E coli (μM) | EPT Y pestis (μM) |
|---|---|---|
| 1 | 5-20 | 20-40 |
| 2 | >200 | 10-30 |
| 3 | 2-40 | 10-30 |
| 4 | >200 | 80-120 |
| 5 | 150-200 | 10-30 |
| 6 | >200 | 50-100 |
| 7 | 5-20 | 20-40 |
| 8 | >200 | NA |
| 9 | >200 | NA |
| 10 | >200 | NA |
| 11 | >200 | NA |
| 12 | >200 | NA |
| 13 | >200 | NA |
| 14 | >200 | NA |
| 15 | 50-100 | NA |
| 16 | NA | NA |
| 17 | NA | NA |
| 18 | NA | NA |
| 19 | NA | NA |
| 20 | NA | NA |
| 21 | 30-50 | 5-15 |
| 22 | 40-60 | 10-30 |
| 23 | 30-50 | 10-20 |
| 24 | 40-60 | 20-30 |
| 25 | >200 | NA |
| 26 | >200 | NA |
| 27 | >200 | NA |
| 28 | >200 | NA |
| 29 | >200 | NA |
| 30 | >200 | NA |
| 31 | 90-110 | 20-40 |
| 32 | 40-60 | 15-25 |
| 33 | 40-60 | 10-20 |
| 34 | 60-80 | 1-10 |
| 35 | >200 | NA |
| 36 | >200 | NA |
| 37 | >200 | NA |
| 38 | >200 | NA |
| 39 | 50-70 | 10-30 |
| 40 | 30-50 | 5-15 |
| 41 | 120-140 | 70-80 |
| 42 | 40-60 | NA |
| 43 | 30-50 | NA |
| 44 | 1-10 | 1-5 |
| 45 | 160-180 | NA |
| 46 | 1-10 | NA |
| 47 | 10-20 | 5-15 |
| 48 | 180-200 | NA |
| 49 | 30-50 | 1-10 |
| 50 | 150-170 | NA |

Approximately 2 μL of compound solution is transferred by serial dilution from column 2 to column 11, resulting in a range of final compound concentrations from about 25 to about 0.049 μg/mL.

Approximately 20 μL of enzyme solution A2 is added into each well of column 1 through 11. Buffer A2 is prepared to contain 50 mM HEPES pH 7.5, 20 mM KCl, 0.02% wt Brij 30, 0.001 mM UDPAG, 0.001 mM PEP, 0.5 mM DTT, and 6 μ/mL MurA.

The plated solutions are incubated for half hour, after which approximately 20 μL of substrate solution B is added to each well, column 1 through 11, to initiate the reaction. Buffer B should be prepared as 2 mM UDPAG, 0.4 mM PEP, 50 mM HEPES pH 7.5, 20 mM KCl, 0.02% wt Brij 30 and 0.5 mM DTT.

After reacting for 8 minutes, 150 μL of Malachite Green is added, the resulting combination incubated for 15 minutes at ambient temperature, and the reaction result is determined by measuring absorbance at 650 nm with a spectrometer.

The data are fit to a curve using Xlfit (ID Business Solutions, Cambridge, Mass.)). The IC₅₀ value can be derived from the curve as the compound concentration that gave 50% inhibition of the enzymatic reaction.

While this invention can be particularly shown and described with references to preferred embodiments thereof, it will be understood by those skilled in the art that various changes in form and details may be made therein without departing from the scope of the invention encompassed by the appended claims.

1. A compound represented by structural formula I-a: 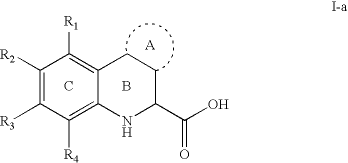
and pharmaceutically acceptable salts, solvates, hydrates, enantiomers, stereoisomers, rotamers, tautomers, diastereomers or racemates thereof,
wherein:
R₁, R₂, R₃, and R₄ are independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C₃ to C₇ cycloalkyl, nonaromatic heterocycle, C₁ to C₄ alkyl, C₁ to C₄ alkoxy, C₁ to C₄ hydroxy alkyl, or C₂ to C₆ alkoxyalkyl;
wherein each R^{b} and R^{d} is independently —H or optionally substituted aryl, aralkyl, heteroaryl, heteroaralkyl, or C₁ to C₄ alkyl, and each R^{c} is independently —H or optionally substituted C₁ to C₄ alkyl, aryl, or aralkyl, or NR^{c}₂ is an optionally substituted nonaromatic heterocycle.

2. The compound of claim 1, wherein, at least two of R₁, R₂, R₃ and R₄ are —H.

3. The compound of claim 2, wherein, one or two of R₁ to R₄ are each independently halogen, —(CO)R^{b}, —(CO)OR^{b}, —(CO)NR^{c}₂, —NR^{c}₂, —NR^{d}(CO)R^{b}, —NR^{d}(CO)OR^{b}, —NR^{d}(CO)NR^{c}₂, —NR^{d}(CO)PhNR^{d}(CO)R^{b}, or optionally substituted phenyl, benzyl, pyridyl, methylpyridyl, or optionally halogenated C₁ to C₄ alkyl or C₁ to C₄ alkoxy; wherein each R^{b}, R^{c}, and R^{d} are independently —H, or optionally substituted C₁ to C₄ alkyl or phenyl, or each NR^{c}₂ is an optionally substituted morpholinyl, piperidyl, or piperazyl.

4. The compound of claim 1, wherein R₁, R₂, R₃, and R₄ are independently —H, —COOR, —COR, —OC(O)R, —CONHR, —NHC(O)R, —COSR, —SC(O)R, —CH═CR, —CH₂CHR, wherein R is defined as biphenyl, substituted biphenyl, substituted aryl, or heteroaryl.

5. The compound of claim 1, wherein R₁, R₂, and R₃ are each a hydrogen.

6. The compound of claim 5, wherein ring A is a cyclopentyl or a cyclopentenyl ring.

7. The compound of claim 6, wherein R₄ is —COOR or —CONHR, wherein R is defined as biphenyl, substituted biphenyl, substituted aryl, or heteroaryl.

8. The compound of claim 7, wherein the substituted biphenyl is substituted with —COOH, —P(O)₃Me₂ or tetrazole.

9. The compound of claim 1, wherein R₁, R₂ and R₃ are each, independently, hydrogen or halogen; Ring A is cyclopentenyl; and R₄ is a —CO₂aryl, —CH₂aryl or —OCH₂aryl group, wherein the aryl groups can be optionally, independently, substituted one or more times with phenyl, halogen, nitro, alkyl, cyano, hydroxyl, alkoxyl, amino or amido.

10. The compound of claim 1, wherein R₁, R₂ and R₃ are each hydrogen or halogen; R₄ is —CO₂phenyl, wherein the phenyl group can be independently substituted one or more times with halogen or phenyl; and A is cyclopentenyl.

11. The compound of claim 1, wherein the compound of formula I-a is selected from the group consisting of the individual compounds provided in Table 1.

12. A method of treating a subject for a bacterial infection, comprising administering to a subject in need of treatment for a bacterial infection an effective amount of a compound of claim 1.

13. The method of claim 12, wherein the subject is a human.

14. The method of claim 12, wherein the infection is caused by a bacterium that expresses phosphoenolpyruvate:UDP-N-acetyl-D-glucosamine 1-carboxyvinyltransferase.

15. The method of claim 12, wherein the infection is caused by a bacterium of a genus selected Allochromatium, Acinetobacter, Bacillus, Campylobacter, Chlamydia, Chlamydophila Clostridium, Citrobacter, Escherichia, Enterobacter, Enterococcus, Francisella, Haemophilus, Helicobacter, Klebsiella, Listeria, Moraxella, Mycobacterim, Neisseria, Proteus, Pseudomonas, Salmonella, Serratia, Shigella, Stenotrophomonas, Staphyloccocus, Streptococcus, Synechococcus, Vibrio, and Yersina.

16. The method of claim 14, wherein the bacterial infection is from Allochromatium vinosum, Acinetobacter baumanii, Bacillus anthracis, Campylobacter jejuni Chlamydia trachomatis, Chlamydia pneumoniae, Clostridium spp., Citrobacter spp., Escherichia coli, Enterobacter spp., Enterococcus faecalis., Enterococcus faecium, Francisella tularensis, Haemophilus influenzae, Helicobacter pylori, Klebsiella spp., Listeria monocytogenes, Moraxella catarrhalis, Mycobacterium tuberculosis, Neisseria meningitidis, Neisseria gonorrhoeae, Proteus mirabilis, Proteus vulgaris, Pseudomonas aeruginosa, Salmonella spp., Serratia spp., Shigella spp., Stenotrophomonas maltophilia, Staphyloccocus aureus, Staphyloccocus epidermidis, Streptococcus pneumoniae, Streptococcus pyogenes, Streptococcus agalactiae, Yersina pestis, and Yersina enterocolitica.

17. A pharmaceutical composition comprising a compound of claim 1.
